(19) United States
(12) Patent Application Publication
(10) Pub. No.: US 2025/0060266 A1
(43) Pub. Date: Feb. 20, 2025

(54) Optical Fiber-Based Medical Device Tracking and Monitoring System

(71) Applicant: Bard Access Systems, Inc., Salt Lake City, UT (US)

(72) Inventor: Shayne Messerly, Kaysville, UT (US)

(21) Appl. No.: 18/936,320

(22) Filed: Nov. 4, 2024

Related U.S. Application Data
(63) Continuation of application No. 15/947,267, filed on Apr. 6, 2018, now Pat. No. 12,140,487.
(60) Provisional application No. 62/483,195, filed on Apr. 7, 2017.

(51) Int. Cl. G01L 5/161; A61B 5/00; A61B 5/01; A61B 5/0215; A61B 5/06; A61B 8/12; A61B 34/20; A61M 25/00; A61M 25/01; G01L 1/24

(52) U.S. Cl. CPC G01L 5/161; A61B 5/0084; A61B 5/066; A61B 5/6852; A61B 8/12; A61B 34/20; A61M 25/0026; A61M 25/0102; G01L 1/242; G01L 1/246; A61B 5/01; A61B 5/0215; A61B 2034/2055; A61B 2034/2061; A61B 2562/0266; A61M 2025/0034; A61M 2025/0166

(57) ABSTRACT

A placement system for tracking, placing, and monitoring a catheter assembly or other medical device inserted into a body of a patient is disclosed. The placement system utilizes optical fiber-based strain sensors to assist with catheter placement. In one embodiment, the placement system comprises a console including a processor and a plurality of optical fiber-based strain sensors included with the catheter. A light source is also included and configured to operably connect with the strain sensors and produce outgoing optical signals incident on the strain sensors. A photodetector is included and configured to operably connect with the strain sensors and receive return optical signals from the strain sensors. A processor is configured to process data from the return optical signals. The data relates to an aspect of the catheter. A user interface such as a display is configured to communicate information relating to the aspect of the catheter.

### CROSS-REFERENCE TO RELATED APPLICATIONS

This application is a continuation of U.S. patent application Ser. No. 15/947,267, filed Apr. 6, 2018, now U.S. Pat. No. 12,140,487, which claims the benefit of priority to U.S. Provisional Application No. 62/483,195, filed Apr. 7, 2017, and titled “Optical Fiber-Based Medical Device Tracking and Monitoring System,” each of which is incorporated herein by reference in its entirety.

### BRIEF SUMMARY

Briefly summarized, embodiments of the present invention are directed to a placement system for tracking, placing, and monitoring a catheter assembly or other medical device inserted into a body of a patient. The placement system utilizes optical fiber-based strain sensors to ascertain information regarding the catheter assembly during and/or after insertion into the patient's body.

In one embodiment, the placement system comprises a plurality of optical fiber-based strain sensors included with the medical device. A laser light source (or other suitable light source) is also included and configured to operably connect with the strain sensors and produce outgoing optical signals incident on the strain sensors. A photodetector is included and configured to operably connect with the strain sensors and receive return optical signals from the strain sensors. A processor is configured to process data from the return optical signals. The data relates to an aspect of the medical device. A user interface such as a display is configured to communicate information relating to the aspect of the medical device.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A and 1B are various block diagram views of a placement system for guiding and placing a medical device into a body of a patient;

FIG. 2 is a simplified view of a patient and the placement system of FIGS. 1A and 1B;

FIG. 3 is a perspective view of a catheter assembly including an optical fiber-based sensor assembly;

FIG. 4 is a side view of a portion of a catheter tube of the catheter assembly of FIG. 3;

FIG. 5 is a cross sectional view of a catheter tube;

FIG. 6 is a cross sectional view of a catheter tube and a stylet inserted therein;

FIG. 7 is a cross sectional view of an optical fiber with a single sensor position region;

FIG. 8 is a cross sectional view of an optical fiber with multiple sensor positions;

FIG. 9 is a perspective view of a stylet;

FIGS. 10A-10C depict various screenshots of the placement system of FIGS. 1A and 1B; and

FIG. 11 is a cross sectional view of a vessel of a body of a patient with a catheter tube disposed therein.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to a placement system for tracking, placing, and monitoring a medical device inserted into a body of a patient. An example of such a medical device is a catheter assembly that is inserted into a vein or other vessel of the patient so as to infuse or aspirate fluids through one or more lumens defined by the catheter for the patient. The system utilizes optical fiber-based strain sensors in one embodiment to ascertain information regarding the catheter or other medical device during and/or after insertion into the patient's body.

In one embodiment, the strain sensors include fiber Bragg grating (“FBG”) sensors distributed along an optical fiber disposed in/on the catheter assembly (or other medical device). An outgoing optical signal produced by a swept laser is incident on each of the FBG sensors in the fiber, which each respond, producing a return optical signal. A processor of the placement system processes the return optical signal with predetermined algorithms to determine the strain and other data of each of the FBG sensors. The data is communicated to a user of the system and can provide information regarding the medical device, its position within the body, the 2-D and 3-D shape of the medical device along its length (e.g., bending, torsion), device orientation (including malposition or device kinking), body temperature, fluid level within the medical device, device pressure, stiffness, and operational load, etc. Such information is presented by the system to the user in real-time to assist in guiding and placing the medical device as desired within the patient. Additionally, measurements may be made by the system post-placement to ensure the medical device is functional and properly placed by interrogating the FBG sensors anew. Further details regarding these and other embodiments are given hereafter.

Note that, though the below discussion focuses on the placement of a catheter assembly into the body of the patient, the placement system described herein can be employed to place a variety of medical devices and components including other elongate and non-elongate medical devices in a variety of locations within the patient body. As such, the principles of the present disclosure should not be considered limiting to what is explicitly described herein. Examples of catheter assemblies and medical devices that may benefit from the present disclosure include a peripherally-inserted central catheter (“PICC”), central venous catheter (“CVC”), urinary catheter, midline catheter, peripheral catheter, an ECG lead, a needle, an NG tube, etc.

In light of the above, the optical fiber-based strain sensor system described above thus serves as one modality in the above-introduced medical device placement system for guiding and placing a medical device within the body of a patient. As such, this modality if also referred to herein as an “optical modality.” Note however that the placement system can also employ additional modalities for improving medical device placement accuracy, in addition to the optical modality introduced above and described in further detail below. In one embodiment an additional ultrasound (“US”) modality is also employed by the system to enable ultrasound-assisted guidance for introducing a catheter assembly (or other medical device) into the patient's vasculature. After such introduction to the vasculature, the optical modality to be described herein can thereafter be employed to guide the catheter assembly to a desired location within the vasculature. These modalities are described in further detail below. In one embodiment, the optical modality alone is employed by the system.

In yet another embodiment, additional modalities may be employed by the system to assist in guiding a catheter assembly (or other medical device) to a desired destination within the body of the patient. These include a tip location/navigation system (“TLS”) modality, wherein magnetically-based tracking of the catheter tip during its advancement through the tortuous vasculature path is employed to detect and facilitate correction of any tip malposition during such advancement; and an ECG modality, wherein ECG signal-based catheter tip guidance is employed to enable tracking and guidance of the catheter tip to a desired position with respect to a node of the patient's heart from which the ECG signals originate. Such ECG-based positional assistance is also referred to as “tip confirmation.” Further details regarding these modalities can be found in U.S. Pat. No. 8,781,555, filed Mar. 2, 2010, and titled System for Placement of a Catheter Including a Signal-Generating Stylet,” which is incorporated herein by reference in its entirety.

Use of the optical modality according to one embodiment enables the catheter placement system to facilitate catheter placement within the patient's vasculature with a relatively high level of accuracy, i.e., placement of the distal tip of the catheter in a predetermined and desired position. In one example implementation, use of the optical modality may result in correct tip placement being confirmed without the need for a confirmatory X-ray. This, in turn, reduces the patient's exposure to potentially harmful x-rays, the cost and time involved in transporting the patient to and from the x-ray department, costly and inconvenient catheter repositioning procedures, etc.

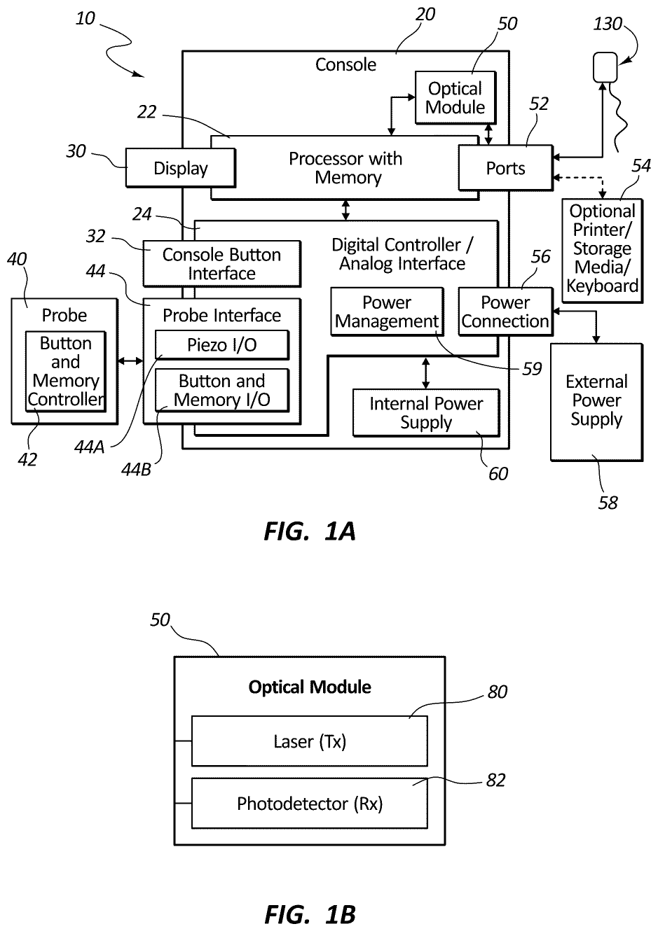

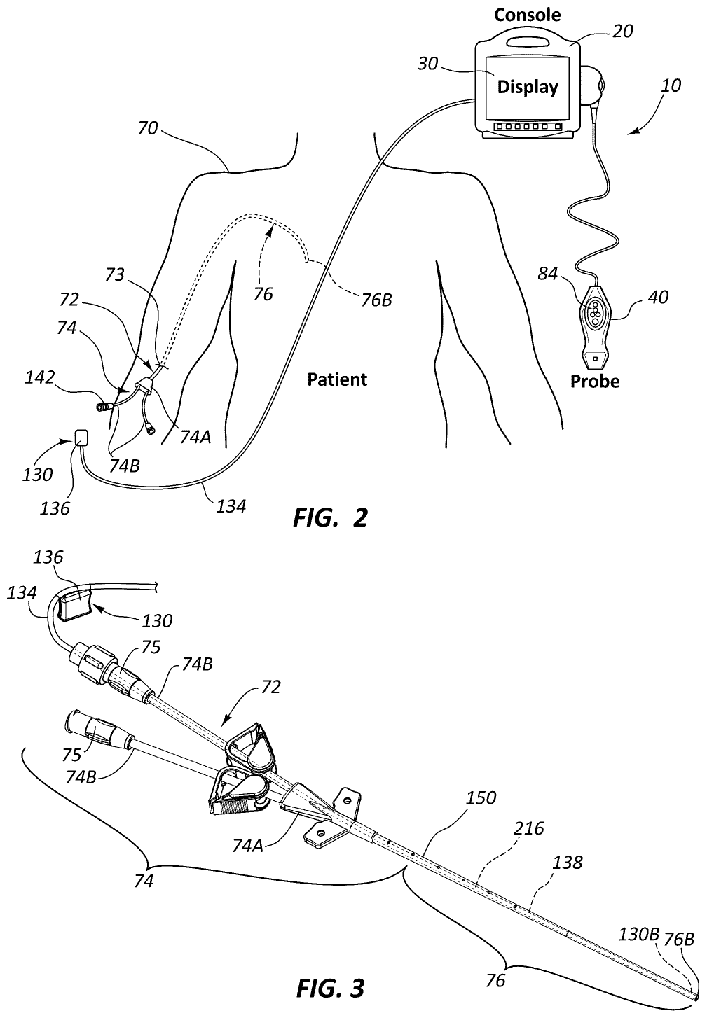

Reference is first made to FIGS. 1A-2, which depict various components of a placement system (“system”), generally designated at 10, configured in accordance with one example embodiment of the present invention. As shown, the system 10 generally includes a console 20, display 30, probe 40, and optical module 50, each of which is described in further detail below.

FIG. 2 shows the general relation of these components to a patient 70 during a procedure to place a catheter 72 into the patient vasculature through a skin insertion site 73. FIG. 2 shows that the catheter 72 generally includes a proximal portion 74 that generally remains exterior to the patient and a distal potion 76 that generally resides within the patient vasculature after placement is complete. The system 10 is employed to ultimately position a distal tip 76A of the catheter 72 in a desired position within the patient vasculature. In one embodiment, the desired position for the catheter distal tip 76A is proximate the patient's heart, such as in the lower one-third (⅓rd) portion of the Superior Vena Cava (“SVC”). Of course, the system 10 can be employed to place the catheter distal tip in other locations. The catheter proximal portion 74 further includes a bifurcation hub 74A that provides fluid communication between the one or more lumens of the catheter 72 and one or more extension legs 74B extending proximally from the bifurcation hub. Note that the bifurcation hub can include one, two, or more fluid paths to fluidly connect the catheter lumens with the corresponding extension legs 74B.

An example implementation of the console 20 is shown in FIG. 2, though it is appreciated that the console can take one of a variety of forms. FIG. 1A shows that a processor 22, including non-volatile memory such as EEPROM for instance, is included in the console 20 for controlling system function during operation of the system 10, thus acting as a control processor. A digital controller/analog interface 24 is also included with the console 20 and is in communication with both the processor 22 and other system components to govern interfacing between the probe 40, optical module 50, and other system components.

The system 10 further includes ports 52 for connection with the optical module 50 and optional components 54 including a printer, storage media, keyboard, etc. The ports in one embodiment are USB ports, though other port types or a combination of port types can be used for this and the other interfaces connections described herein. A power connection 56 is included with the console 20 to enable operable connection to an external power supply 58. An internal battery 60 can also be employed, either with or exclusive of an external power supply. Power management circuitry 59 is included with the digital controller/analog interface 24 of the console to regulate power use and distribution.

The display 30 in the present embodiment is integrated into the console 20 and is employed as a user interface to display information to the clinician during the catheter placement procedure. In another embodiment, the display may be separate from the console. As will be seen, the content depicted by the display 30 changes according to which mode the catheter placement system is in: optical, US, or other modality. In one embodiment, a console button interface 32 and buttons included on the probe 40 can be used to immediately call up a desired mode to the display 30 by the clinician to assist in the placement procedure. In one embodiment, information from multiple modes, such as optical and US, may be displayed simultaneously. Thus, the single display 30 of the system console 20 can be employed for ultrasound guidance in accessing a patient's vasculature and optical modality-based guidance during catheter advancement through the vasculature. In one embodiment, the display 30 is an LCD device.

The probe 40 is employed in connection with the first modality mentioned above, i.e., ultrasound (“US”)-based visualization of a vessel, such as a vein, in preparation for insertion of the catheter 72 into the vasculature. Such visualization gives real time ultrasound guidance for introducing the catheter into the vasculature of the patient and assists in reducing complications typically associated with such introduction, including inadvertent arterial puncture, hematoma, pneumothorax, etc.

The handheld probe 40 includes a head that houses a piezoelectric array for producing ultrasonic pulses and for receiving echoes thereof after reflection by the patient's body when the head is placed against the patient's skin proximate the prospective insertion site 73 (FIG. 2). The probe 40 further includes a plurality of control buttons, which can be included on a button pad. In the present embodiment, the modality of the system 10 can be controlled by the control buttons, thus eliminating the need for the clinician to reach out of the sterile field, which is established about the patient insertion site prior to catheter placement, to change modes via use of the console button interface 32.

As such, in one embodiment a clinician employs the first (US) modality to determine a suitable insertion site and establish vascular access, such as with a needle or introducer, then with the catheter. The clinician can then seamlessly switch, via button pushes on the probe button pad 82, to the optical modality without having to reach out of the sterile field. Again, the optical modality can be used to assist in advancement of the catheter 72 through the vasculature toward an intended destination.

FIG. 1 shows that the probe 40 further includes button and memory controller 42 for governing button and probe operation. The button and memory controller 42 can include non-volatile memory, such as EEPROM, in one embodiment. The button and memory controller 42 is in operable communication with a probe interface 44 of the console 20, which includes a piezo input/output component 44A for interfacing with the probe piezoelectric array and a button and memory input/output component 44B for interfacing with the button and memory controller 42.

The handheld ultrasound probe 40 is employed to enable US visualization of the peripheral vasculature of a patient in preparation for transcutaneous introduction of the catheter. In the present example embodiment, however, the probe is also employed to control functionality of other modalities of the system 10, including the optical modality, when navigating the catheter toward its desired destination within the vasculature as described below. Again, as the probe 40 is used within the sterile field of the patient, this feature enables functionality of any one of the modalities to be controlled entirely from within the sterile field. Thus the probe 40 is a multi-purpose device, enabling convenient control of both US and other modality functionality of the system 10 from the sterile field. Note that additional or fewer components can be included with the system 10 than what is shown and described herein.

As mentioned, the system 10 includes the optical module 50, which is shown in further detail ion FIG. 1B. As shown, the optical module 50 includes an optical transmitter, such as a laser 80, configured to produce outgoing optical signals incident on a plurality of optical fiber-based strain sensors (described further below) of the catheter assembly. The optical module 50, as well as an optical receiver, such as a photodetector 82, configured to receive return optical signals from the optical fiber-based strain sensors of the catheter assembly. In one embodiment, the laser 80 is a tunable swept laser and both the laser 80 and the photodetector 82 are operably connected to the processor 22 of the system 10 (FIG. 1A), which governs their operation. In another embodiment, another suitable light source can also be employed in addition to a laser, including semi-coherent light sources, LED light sources, etc.

FIG. 1A also shows that the optical module in the present embodiment is operably connected to a stylet 130, which includes an optical fiber and the plurality of strain sensors, as will be described further below. The stylet 130 is configured to be removably received within the catheter 72 during the procedure to place the catheter into the vasculature of the patient and enable the system 10 to guide the catheter distal tip 76B to a desired destination within the body. Note, however, that the optical fiber and strain sensors can be configured in other ways other than a stylet, some of which are discussed further below. Note further that the optical module 50 can include other components and be included within the system 10 in other configurations apart from what is shown and described herein.

FIG. 2 shows the stylet 130 in place within the catheter 72 as the catheter is being inserted into the patient 70 with the assistance of the system 10. FIGS. 2 and 3 depict further details regarding the stylet 130 and the catheter 72. As shown, the catheter 72 includes an elongate catheter tube 150 defining one or more lumens 216 extending between proximal and distal ends of the catheter tube, which are in communication with the corresponding extension legs 74B via the bifurcation hub 74A, as described further above. In the illustrated embodiment the catheter 72 includes two lumens 216 and two corresponding extension legs 74B, though other numbers of lumens and extension legs are possible. Luer connectors 75 are included on the proximal ends of the extension legs 74B.

Along with FIGS. 2 and 3, reference is made to FIG. 9, which together depict details regarding the stylet 130 and the catheter 72. As shown, the stylet 130 is an elongate device and includes a system connector 132 on its proximal end 130A to enable the stylet to operably connect with the console 20 (FIG. 2) or other suitable component of the system 10 via a threading (or other suitable) engagement. A tether 134 distally extends between the system connector 132 to a catheter connector 142 configured to threadably engage (or otherwise connect with) the luer connector 75 of one of the extension legs 74B of the catheter 72, as seen in FIG. 3. A fiber-bearing portion 138 of the stylet 130 extends distally from the catheter connector 142 to a distal end 130B of the stylet. A handle 136 is included with the tether 134 to assist with manipulation of the stylet 130 by the user during system operation.

In greater detail, the fiber-bearing portion 138 includes, as seen in FIG. 6, an elongate core wire 138A with an optical fiber 140 disposed in a longitudinal notch defined along an outer surface thereof. So configured, the core wire 138A and optical fiber 140 extend distally from the catheter connector 142 to the distal end of the stylet 130 (FIG. 9). The optical fiber 140 is secured within the notch of the core wire 138A by potting 214 (such as an adhesive), or by other suitable mode. The core wire 138A includes a suitable, flexible material, including stainless steel for instance. FIG. 6 shows the fiber-bearing portion 138 disposed in a lumen 216 defined by a wall 210 of a single-lumen catheter tube 150, though it is appreciated that the size, shape, and other configuration of the fiber-bearing portion 138 can vary from what is shown and described herein in order to accommodate catheters, lumens, and medical devices of differing configurations.

As shown in FIG. 2, the stylet 130 is inserted into the catheter 72 during use of the system 10 to place the catheter 72 in the body of the patient 70. The stylet 130 is shown with the fiber-bearing portion 138 disposed in the lumen of the catheter 72 and the tether 134 extending from the fiber-bearing portion to the console 20 such that the stylet is operably connected to the system 10 (via the stylet system connector 132). In this way, outgoing optical signals produced by the laser 80 (FIG. 1B) of the optical module 50 and return optical signals to be received by the photodetector 82 can travel to and from the optical fiber 140 (FIG. 6) and its corresponding strain sensors, as will be discussed further below.

FIG. 3 depicts the manner of operable connection of the stylet with the catheter 72. As shown, the fiber-bearing portion 138 is disposed within one of the lumens 216 of the catheter tube 150 such that the distal end of the stylet 130B—and the distal end of the fiber-bearing portion and accompanying optical fiber 140—is substantially co-terminal with the distal tip 76B of the catheter. The fiber-bearing portion 138 extends proximally up the lumen 216, through the bifurcation hub 74A and corresponding extension leg 74Bto the catheter connector 142, which is shown threadably attached to the luer connector 75 of the extension leg. The tether 134 is further shown extending from the catheter 72 to operably connect with the console 20, as shown in FIG. 2. This connective configuration is used during the procedure to insert the catheter 72 into the body of the patient 70, and in one embodiment the stylet is pre-loaded into the catheter during time of catheter manufacture or prior to commencement of the insertion procedure. It is appreciated that the stylet 130, the fiber-bearing portion 138, and the optical fiber 140 can be configured in other ways while still enabling the desired functionality of the system 10 to be performed. In yet another embodiment, the optical fiber 140 need not be substantially co-terminal with the distal tip 76B of the catheter 72, but can terminate proximal or distal thereto, as may be appreciated by one skilled in the art. As will be seen, the strain sensors of the optical fiber 140 included with the fiber-bearing portion 138 of the stylet 130 enable the catheter 72 to be tracked during its advancement through the patient vasculature.

Note further that, though described herein as a stylet, in other embodiments a guidewire or other catheter guiding apparatus could include the components and functionality described herein. Indeed, it should appreciated that “stylet” as used herein can include any one of a variety of devices configured for removable placement within a lumen of the catheter (or other portion of a medical device) to assist in placing a distal end of the catheter in a desired location within the patient's vasculature. Also, note that other connection schemes between the stylet 130 and the system 10/console 20 can also be used without limitation.

Note that a sterile drape is often positioned over the patient 70 during the catheter insertion procedure in order to define the majority of a sterile field: areas above the drape are sterile, while areas below (excluding the insertion site and immediately surrounding region) are non-sterile. For instance, areas and components above the drape and proximate to the insertion site 73 (including the catheter 72, the stylet 130, and tether 134) are included in the sterile field, while areas and components below the drape, including the patient's chest and regions immediately surrounding the patient 70 are considered a non-sterile field. In cases where the tether 134 of the stylet 130 needs to penetrate the drape in order to pass between the sterile and non-sterile fields, suitable connection nodes can be included with the stylet in order to pierce the drape while not compromising the sterile field. U.S. Pat. No. 8,781,555, incorporated by reference above, includes various examples of drape-piercing embodiments that can be employed to enable the stylet to acceptably pass through the drape while still enabling outgoing and return optical signals, as well as other signals, to pass between the optical fiber 140 and the console 20.

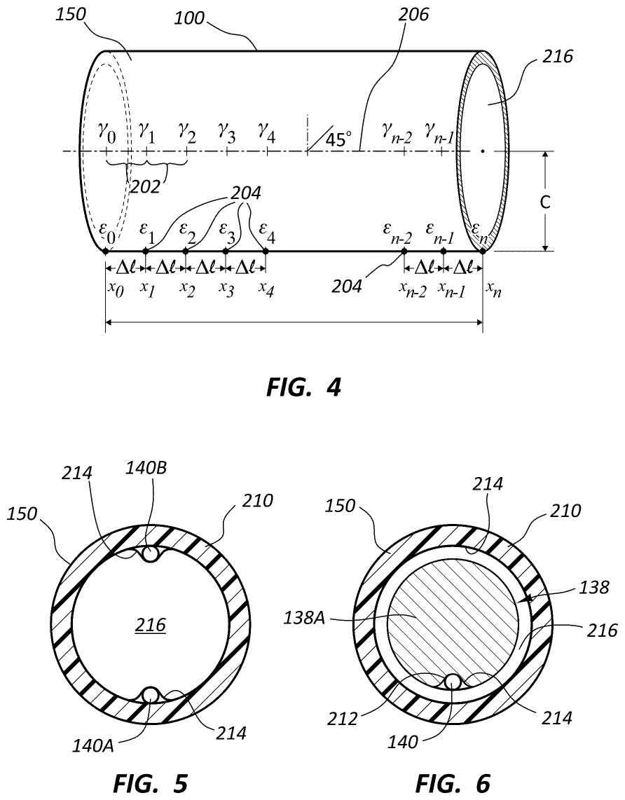

FIG. 4 generally depicts a portion, or structure 100, of the catheter tube 150 of the catheter 72 to depict the spatial relationship between the catheter tube and the strain sensors included with the optical fiber 140 discussed above. As shown, the structure 100 is subdivided into a plurality of sections 202 (also designated with γᵢ (i=0, 1, 2, 3, . . . n) such that the sections are designated as γ₀, γ₁, . . . , γₙ), each section having a length Δl, wherein strain sensors 204 are positioned at the junction of each adjacent section 202, as well as at the beginning and end of the sections, designated in FIG. 4 as xᵢ, thus at x₀, x₁ . . . , xₙ. The strain sensors 204 are positioned a distance c from a neutral axis of 206 of the catheter tube 150. When operative, the strain sensors 204 are capable of each detecting a corresponding strain & such that strain co is detected at strain sensor 204 positioned at x₀ at the junction for section γ₀, etc. U.S. Pat. No. 7,715,994, incorporated herein by reference in its entirety, gives further details regarding the strain sensors and their spatial distribution as depicted in FIG. 4.

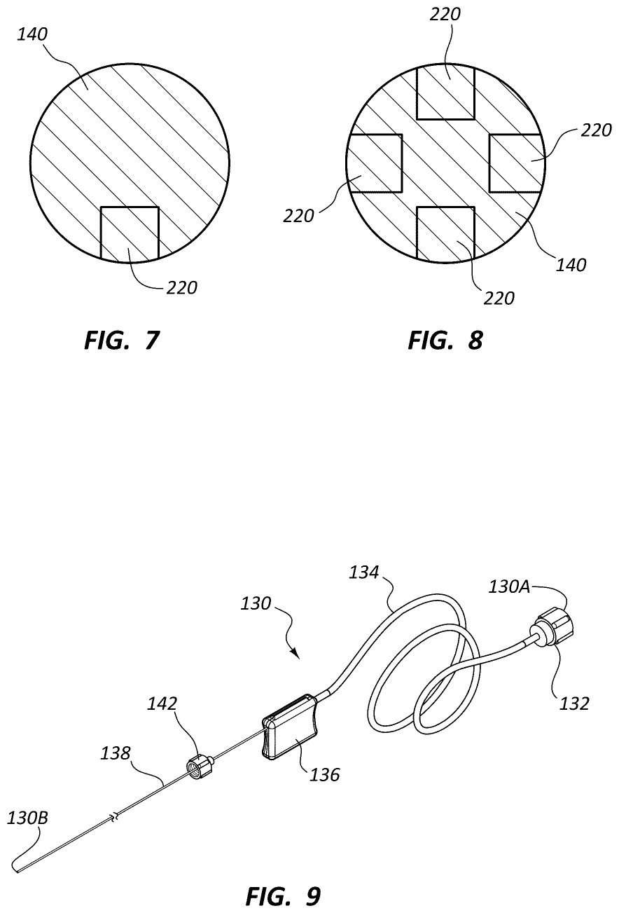

FIG. 7 shows that, in one embodiment, a predetermined sensor position region 220 is defined within the optical fiber 140, wherein the sensors 204 are disposed along the longitudinal length of the optical fiber. In another embodiment, multiple sensor position regions 220 can be defined within the single optical fiber 140, as shown in FIG. 8, for instance, such that distinct series, or channels, of strain or other sensors can be included on a single optical fiber. These multiple, non-colinear channels of strain sensors can be employed for differing purposes, such as one or more series to indicate medical device position with another series to monitor temperature along the medical device, for instance. These and other strain sensor position configurations with the optical fiber are therefore contemplated.

The strain sensors 204 in one embodiment can be included with an optical fiber that is in turn included in a fiber-bearing of a stylet, such as the stylet 130 described above in connection with FIGS. 3, 6, and 9. In another embodiment, the strain sensors 204 are included with an optical fiber that is permanently incorporated into the catheter tube 150, such as is seen in FIG. 5. Many other possible strain sensor implementations are possible, including other types of fiber optic sensors.

In the present embodiment, the strain sensors 204 are configured as fiber Bragg grating (“FBG”) sensors. The FBG sensors 34 are each configured to detect strain of the optical fiber at each sensor location, thus enabling the shape and movement of the optical fiber, and thus the catheter tube 150 with which it is included, to be detected and determined as the catheter is inserted into the body of the patient 70, as explained immediately below.

In greater detail, once the catheter 72 has been introduced into the patient vasculature via the insertion site 73 (FIG. 1) the optical modality of the system 10 can be employed to advance the catheter distal tip 76A toward its intended destination proximate the SA node, in the current embodiment, noting that other intended destinations are also possible. The catheter 72 carries within one of its lumens 216 the stylet 130 including the fiber-bearing portion 138, which in turn includes the optical fiber 140 with its strain sensors 204 (e.g., the FBG sensors in the present embodiment). As the stylet-loaded catheter 72 is advanced toward the patient's heart, outgoing optical signals are produced by the laser 80 of the optical module 50 (FIGS. 1A, 1B), which optical signals propagate distally through the optical fiber 140 via the stylet tether 134 to interrogate each of the FBG-type sensors 204, resulting in return optical signals from the sensors. The return optical signals from the FBG-type sensors 204 are received by the photodetector 82 of the optical module after travelling proximally through the optical fiber 140 and the stylet tether 134, which are then forwarded to the processor 22. The outgoing optical signal interrogation and return optical signal process is iterated at a scan rate to provide real-time monitoring. In one embodiment scan rates of 24 scans per second are employed, though other rates are possible. Specified algorithms and processes are followed to determine from the return optical signals the strain data from each of the FBG-type sensor 204. Further information regarding the algorithms and processes referred to immediately above, including serial multiplexing, suitable wavelength division multiplexing (“WDM”), and optical frequency domain reflectometry (“OFDM”) processes with regard to a fiber optic sensing system (“F.O.S.S.”) is described in: U.S. Pat. Nos. 7,520,176; 7,715,994; 8,700,358; and 8,909,040, each of which is incorporated herein by reference in its entirety.

In one embodiment and in light of the above, therefore, a method for placing a catheter assembly (or other medical device) into a body of a patient includes stages: propagating an outgoing optical signal to a plurality of optical fiber-based strain sensors included with the catheter assembly; receiving a return optical signal from the strain sensors; and processing the return optical signal received from the strain sensors to derive data relating to the medical device. These stages are successively repeated while inserting the catheter assembly into the body of the patient so as to guide the catheter assembly to its intended destination.

This above-described process results in specified strain data to be correlated along the sensor-equipped length of the optical fiber 140. In turn, given the disposal of the optical fiber 140 within the lumen 216 of the catheter tube 150, the strain data enables information regarding the two- and three-dimensional shape of the catheter tube 150 within the patient of the patient 70 to be ascertained, given that the detected strain-related displacement, bending, pressure, and torsion/twisting of the optical fiber 140 correlates to the catheter tube itself in which the optical fiber is disposed. This and other informational aspects relating to the data collected from the sensors 204 can be communicated to the user of the system 1, via the display 30 and/or other suitable user interface mode, to assist the user in knowing the location, orientation and shape of the catheter 72 within the patient vasculature, thus enabling the user to advance the catheter distally and position the distal tip 76B of the catheter in a desired location therein, in part by the fact that the distal end of the sensor-equipped optical fiber 140 is substantially co-terminal with the distal tip of the catheter within the vasculature, in the present embodiment. The process of sending and receiving outgoing and return optical signals, respectively, occurs iteratively during system operation such that position, orientation, and shape information relating to the catheter 72 is continuously received by the user via the system 10 during the catheter placement procedure.

Once it has been positioned as desired using the system 10 as just described, the catheter 72 may be secured in place and the stylet 130 removed from the catheter lumen. It is noted here that the stylet may include one of a variety of configurations in addition to what is explicitly described herein. In one embodiment, the stylet can attach directly or indirectly to the console. In another embodiment, the optical fiber of the stylet can be integrated into the catheter structure itself, thus eliminating the need for the stylet in bearing the optical fiber and included strain sensors. FIG. 5 gives one example of such an embodiment, wherein the catheter tube 150 includes first and second optical fibers 140A, 140B that are each affixed to an inner surface of the tube wall 210 that defines the lumen 216. A potting 214, such as an adhesive, is used to adhere the optical fibers 140A, 140B to the tube wall 210. One, two, or more optical fibers can be included in the catheter tube in this manner. Also, in one embodiment, the optical fiber can incorporated into the catheter wall itself, such as via a co-extrusion process, for instance. These and other variations are therefore contemplated.

Note that in one embodiment the console 20 includes the electronic components, such as the processor 22 (FIG. 1), necessary to process the return optical signals received by the photodetector 82 via the stylet 130 or other suitable structure including the optical fiber 140 and sensors 204. In another embodiment, this functionality can be included in another system component including, for example, the optical module 50.

In the present embodiment, the position, orientation, shape, and other information regarding the catheter 72, as provided by the optical fiber-based sensors 204 and as described above is communicated to the user of the system 10 to assist with placing the distal tip 76B (or other portion of the catheter) at a desired location within the patient vasculature/body, such as the lower ⅓rd of the superior vena cava. In the present embodiment, such information is depicted on the display 30, included on the console 20 as part of the system 10, though it can be configured in other ways as well, including as a separate component in one embodiment. As has been described further above, the functionality of the display 30 can be controlled by control buttons 84 included on the handheld probe 40 (FIGS. 1A, 2), thus eliminating the need for the clinician to reach out of the sterile field (such as touching the button interface 32 of the console 20) to change modes. Thus, in the present embodiment the probe 40 is employed to also control some or all functionality of the system 10. Note that the button interface 32 or other input configurations can also be used to control system functionality. Also, in addition to the visual display 30, aural information, such as beeps, tones, etc., can also be employed by the system to assist the clinician during catheter placement. Moreover, the buttons included on the probe 40 and the console button interface 32 can be configured in a variety of ways, including the use of user input controls in addition to buttons, such as slide switches, toggle switches, electronic or touch-sensitive pads, etc.

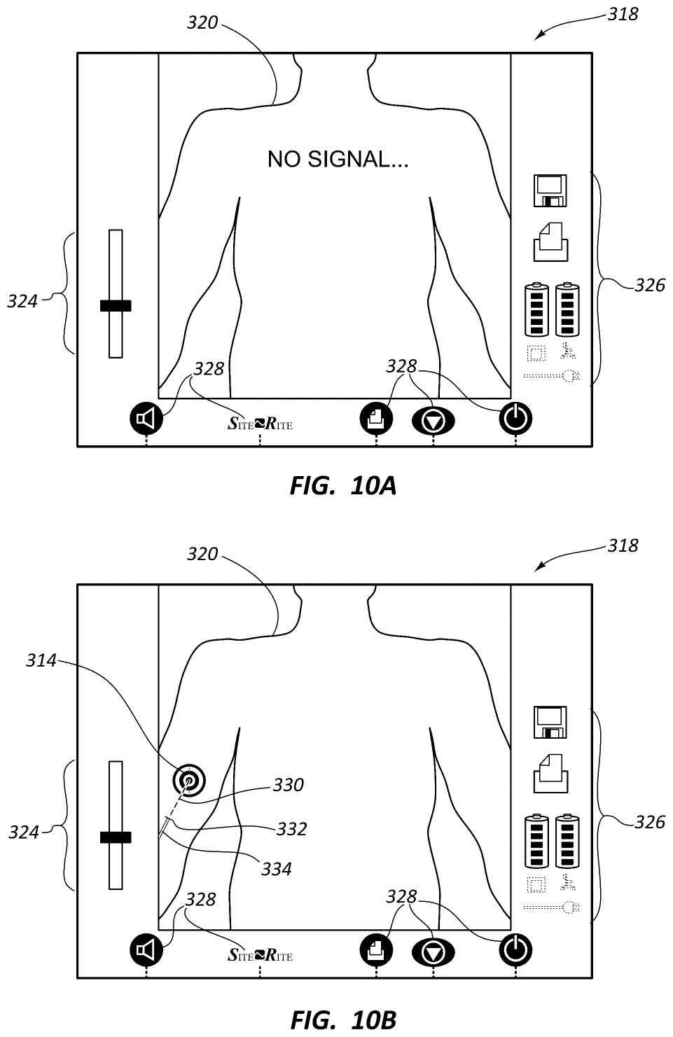

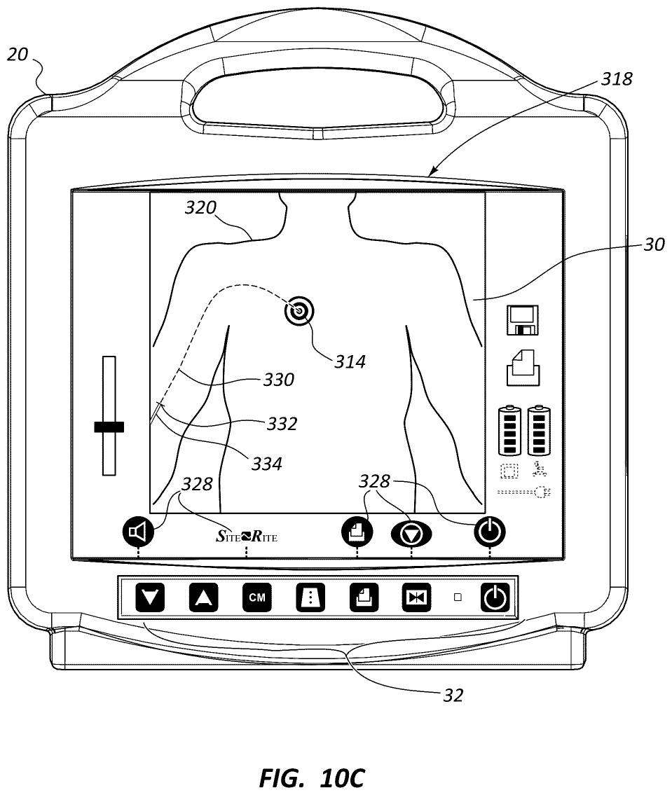

FIG. 10A shows an example screenshot 318 as depicted on the display 30 while the system 10 is in operation. A representative body image 320 is shown. Other information is provided on the display screenshot 318, including a depth scale indicator 324, status/action indicia 326, and icons 328 corresponding to the button interface 32 included on the console 20 (FIG. 1A). Though the icons 328 in the present embodiment are simply indicators to guide the user in identifying the purpose of the corresponding buttons of the button interface 32, in another embodiment the display can be made touch-sensitive so that the icons themselves can function as button interfaces and can change according to the mode the system is in.

During initial startup and preparation for insertion of the catheter 72 into the patient vasculature, the display screenshot may indicate “no signal,” as seen in FIG. 10A, indicating that no data is being returned from the sensors 204 of the optical fiber 140. In FIG. 10B, a distal portion of the catheter 72 has been inserted into the body of the patient 70, which is represented in the screenshot 318 by a relatively short path of travel 330 distal to a depicted insertion site 332 (which corresponds to the actual insertion site 73 of FIG. 2) on the display 30. A still-external portion 334 of the catheter is also shown on the screenshot 318 of FIG. 10B, proximal to the depicted insertion site 332. Temperature-based differences as detected in data received from the return optical signals enables the system 10 to determine at which point along the length of the optical fiber 140 the catheter tube 150 has entered into the body 70 of the patient in the present embodiment, thus enabling the system to depict the location of the insertion site 332. In like manner, the internal path of travel 330 and the external portion 334 can be depicted on the display. A position icon 314 is also depicted at the detected distal end of the sensor-equipped optical fiber 140, corresponding to the distal tip 76B of the catheter 72, in the present embodiment.

In FIG. 10C, the catheter 72 has advanced proximate a desired position within the patient vasculature, i.e., proximate the lower ⅓rd portion of the SVC in the present embodiment, as indicated by the position icon 314 on the screenshot body image 320. This is indicated by the relatively longer internal path of travel 330 with respect to the insertion site 332. The system 10 is further able to detect and depict any bends or changes in direction in the catheter via processing of the return optical signals from the various sensors 204 along the length of optical fiber disposed in one of the lumens 216 of the catheter tube 150 (FIG. 3). As such, position, shape, and orientation of the catheter 72 is determined by the system 10 and communicated to the clinician via the display 30, thus enabling the clinician to accurately guide the catheter to the desired position within the vasculature.

In one embodiment it is appreciated that the mapping of the catheter as an image on the body image 320 to indicate the actual location of the catheter 72 within the actual body of the patient 70 is enabled by knowledge of the insertion site of the catheter (i.e., a reference location)—here represented by the insertion site 332 of FIG. 10C, the length of the portion of the catheter found inside the patient body (represented by the internal portion of the catheter 330), and the shape/displacement of the catheter as detected by the optical fiber-based sensors 204 disposed within the catheter.

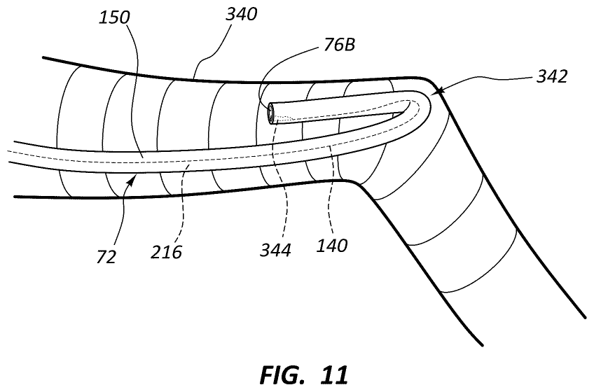

FIG. 11 depicts a scenario where optical modality of the system 10 can assist a clinician in detecting a malposition scenario for the catheter 72 within the vasculature. As shown, the catheter tube 150 is disposed within a vessel 340 of the patient 70 in such a way as to undesirably double back on itself and form a kink region 342. Such kinks are possible during insertion of the catheter 72 into the body. The optical fiber 140, including the plurality of FBG-type sensors 204, is also shown running along the length of the lumen defined by the catheter tube 150. Return optical signals from the sensors 204 in the kink region 342 during system operation will indicate the strain in the optical fiber 140 caused by the kink. After processing by the system 10, information relating to the kink can be communicated to the clinician via the display 30 or by other suitable means. In one example, for instance, the clinician can view the catheter tube doubling back on itself as depicted on the display. Once the kink condition has been identified, the clinician can correct the kink and proceed with catheter insertion.

FIG. 11 further shows an obstruction 344, such as a thrombus or fibrin sheath, present proximate the distal end 76B of the catheter tube 150. Such obstructions are also detectable by sensors 204 of the optical fiber 140 as part of the optical modality of the system 10. Once the obstruction condition has been communicated to the clinician, corrective measures may be undertaken to clear the obstruction from the catheter tube 150.

It should be appreciated that the use of optical fiber-based sensors can be employed to confirm that a previously inserted catheter assembly (or other medical device) is still positioned as desired within the body of the patient. In one embodiment, this is achieved by feeding a stylet including an optical fiber-based series of strain sensors distally within a lumen of the catheter until reaching the distal tip of the catheter. Strain and other measurements may then be performed by the sensors disposed within the catheter lumen, thus enabling the system with which the sensors are operably connected to determine the shape, orientation, and position of the catheter tube within the vasculature of the patient, including the termination point of the catheter distal tip. This enables a clinician to ensure the catheter is still positioned at the desired location.

In addition to strain, stress, and torsion measurements that enable the two- and three-dimensional shape, position, and orientation of the catheter or other medical device to be determined while in the patient body, the optical modality described herein can detect and communicate other information to the clinician via the system 10 using the data provided by the sensor interrogation by the outgoing optical signals and the resultant return optical signals. Such information includes temperature, pressure, stiffness, strength, operational load on the catheter tube, liquid level within the lumen 14, blood oxygen level, magnetic field presence, etc. Further, it is appreciated that, in light of the above disclosure, other conditions relating to the catheter may be detected, including malposition/misdirection during catheter advancement including contralateral and IJ placement, arterial vs. venous placement, venous pressure and core body temperature, confirmation of the catheter staying within the true lumen of the vein or other vessel in the case of navigation through chronic total occlusion or other vessel blockage scenarios, etc.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A catheter placement system, comprising:
a catheter including a lumen extending between a proximal end and a distal end of the catheter;
a stylet removably inserted into the lumen of the catheter, the stylet including a core wire and an optical fiber secured in a longitudinal notch of the core wire, and the optical fiber including a first plurality of optical-fiber-based strain sensors disposed along a first longitudinal portion of the optical fiber and a second plurality of optical-fiber-based strain sensors disposed along a second longitudinal portion of the optical fiber;
a light source operably connected to the stylet, the light source configured to produce outgoing optical signals incident on the first plurality of optical-fiber-based strain sensors and the second plurality of optical-fiber-based strain sensors;
a photodetector operably connected to the stylet, the photodetector configured to receive return optical signals from the first plurality of optical-fiber-based strain sensors and the second plurality of optical-fiber-based strain sensors;
a console including memory and a processor configured to:
process optical data from the return optical signals; and
map the catheter in accordance with various aspects of the stylet including a location of the stylet within a body of a patient; and
a display configured to display information relating to the various aspects of the stylet including an image of the catheter mapped on the body of the patient.

2. The catheter placement system of claim 1, wherein the stylet includes a catheter connector and a console connector respectively configured to connect to a Luer connector of the catheter and a port of the console.

3. The catheter placement system of claim 2, the stylet further including:
a tether between the catheter connector and the console connector; and
a handle with the tether to assist with manipulation of the stylet by a user.

4. The catheter placement system of claim 1, wherein the light source is a tunable swept laser, the laser and the photodetector disposed in an optical module.

5. The catheter placement system of claim 1, wherein the light source is configured to iteratively emit the outgoing optical signals and the photodetector is configured to iteratively receive the return optical signals.

6. The catheter placement system of claim 1, wherein the first plurality of optical-fiber-based strain sensors is included on a first channel of the optical fiber and the second plurality of optical-fiber-based strain sensors is included on a second channel of the optical fiber, the first and second channels of the optical fiber being non-colinear with each other.

7. The catheter placement system of claim 1, wherein the first plurality of optical-fiber-based strain sensors and the second plurality of optical-fiber-based strain sensors include fiber Bragg gratings.

8. The catheter placement system of claim 1, wherein the various aspects of the stylet include the location of the stylet within the body of the patient and at least another aspect of the stylet selected from a two-dimensional shape, a three-dimensional shape, a position or orientation within the body of the patient, a pressure, and a temperature of the stylet.

9. The catheter placement system of claim 1, wherein the various aspects of the stylet include the location of the stylet within the body of the patient and a temperature of the stylet.

10. The catheter placement system of claim 9, wherein differences in the temperature of the stylet along a length of the stylet enable the catheter placement system to determine when a point along the length of the stylet has entered the body of the patient.

11. The catheter placement system of claim 10, wherein the differences in the temperature of the stylet along the length of the stylet enable the catheter placement system to display a location of the insertion site on the display.

12. The catheter placement system of claim 1, wherein the stylet is substantially coterminal with the catheter for indicating a location of the catheter within the body of the patient.

13. The catheter placement system of claim 1, wherein the information relating to the various aspects of the stylet assists a user of the catheter placement system in guiding the stylet and the catheter to a desired location within the body of the patient for placement of the catheter.

14. The catheter placement system of claim 1, further comprising an ultrasound probe configured for ultrasound-based visualization of a blood vessel of the patient in preparation for insertion of the catheter into the blood vessel.

15. The catheter placement system of claim 14, wherein the ultrasound probe provides an ultrasound modality for the insertion of the catheter into the blood vessel, and the stylet in the catheter provides an optical modality for guiding placement of the catheter within the body of the patient.
